(19) United States
(12) Patent Application Publication
(10) Pub. No.: US 2024/0066195 A1
(43) Pub. Date: Feb. 29, 2024

(54) VOICE COIL VACUUM MOTOR WITH VIBRATION AND/OR RELEASE

(71) Applicant: LANSINOH LABORATORIES, INC., Alexandria, VA (US)

(72) Inventors: Frank Tinghwa WANG, Taipei City (TW); Rush Lloyd Bartlett, II, Alexandria, VA (US)

(21) Appl. No.: 18/261,123

(22) PCT Filed: Jan. 28, 2022
(86) PCT No.: PCT/US2022/014344

§ 371 Date: Jul. 12, 2023

Related U.S. Application Data
(60) Provisional application No. 63/142,796, filed on Jan. 28, 2021.

(51) Int. Cl. A61M 1/06; A61M 1/00; H02K 33/16

(52) U.S. Cl. CPC A61M 1/069; A61M 1/80; H02K 33/16

(57) ABSTRACT

An example vacuum motor device for facilitating milk extraction from a breast of a user can include: a first chamber; a second chamber coupled to the first chamber; and a voice coil motor configured to cause air to flow into the second chamber and out of the first chamber during a breast pumping cycle to create suction for extracting the milk.

### RELATED APPLICATION(S)

This application is being filed on Jan. 28, 2022, as a PCT International Patent application and claims the benefit of and priority to U.S. Patent application Ser. No. 63/142,796 filed on Jan. 28, 2021, the entire disclosure of which is incorporated by reference in its entirety.

### FIELD

The present application is directed to devices, systems and methods for facilitating the collection of breast milk.

### BACKGROUND

Breastfeeding is the recommended method to provide nutrients to a newborn child for the first year of life. Many mothers, however, return to work soon after giving birth, have difficulty breastfeeding their newborns, or have challenges breastfeeding for other reasons. As a result, many mothers rely on breast pumping to express their breast milk and use bottles to feed their newborns. Since a mother might need to pump as often as eight times a day to maintain her milk supply and/or prevent breast engorgement, each breast pumping session should also be as efficient as possible—i.e., emptying as much milk from the breast as possible, in the shortest amount of time.

Breast pumps operate by applying a suction on the breast for a short period of time, during which a small amount of milk is expressed. The breast pump then releases the suction and repeats the cycle of on suction/off suction until the breast is empty. The amount of vacuum applied to the breast during one cycle of on suction/off suction, referred to as a waveform, is controlled by the breast pump by adjusting the applied voltage and/or current to an internal vacuum motor and solenoid, to mimic the baby feeding on the breast. Typical breast pumps allow the mother to adjust the cycle speed and the amount of suction, in an attempt to maximize efficiency of the pump.

Therefore, it would be ideal to have a breast pump that worked efficiently to prevent breast engorgement. Ideally, such a breast pump would empty as much milk from the breast as possible, in a short amount of time, while being discrete. Additionally, such a breast pump would also ideally be easy to adjust for an individual woman's specific needs. At least some of these objectives are addressed by the following disclosure.

### SUMMARY

The present document describes various devices and techniques to generate a vacuum, and/or release vacuum and/or generate a diverse set of vibration patterns. This may be done with one or more of a variety of components including at one of a voice coil actuator, solenoid, DC electric motor, piezoelectric motor, mechanical valve, transducer, or other system. For configurations with a vacuum generator such as a voice coil actuator alone, or voice coil or other vacuum generator in combination with other components, it can be desirable to enable users to tailor the vacuum including custom performance of the vibration and/or enable vibrations to be present or absent in ways not currently enabled by the state of the art.

One embodiment of this application describes an improved breast pump device and method that allows for increased milk volume flow rates and/or increased pump efficiency. The device and method involve applying vibrations to the breast during the breast pump cycle (or “waveform”), to increase the volume flow rate of expressed milk for a given cycle speed and suction level. The breast pump waveform with added vibrations according to the present disclosure is often referred to herein as a “vibratory waveform.” The vibratory waveform helps the breast pump empty milk from the breast more completely and/or in a shorter time than would occur from simply adjusting the breast pump's cycle speed and/or suction level. Creating a vibratory waveform may also reduce the time to let down, the reflex that leads to the release of breast milk. In any given pumping method example, the vibratory waveform may be applied, and the pump's cycle speed and/or suction level may also be adjusted. Alternatively, the vibratory waveform may be applied (and have advantageous results) without any adjustment of cycle speed or suction level.

In various embodiments, the breast pump applies vibration to the breast through small oscillations in the suction pattern as the vacuum is reduced, held, and/or released, as part of the pump cycle. The vibrations may facilitate improved let down and reduce the shear stress of milk against the inner walls of the milk ducts, to help increase the volume flow rate of milk flowing out of the milk duct. The vibration feeling is most pronounced when the suction is increased and decreased in a rapid cyclical manner.

The vibratory waveform can be generated in a breast pump system using a variety of devices and methods. In some embodiments, a vibratory device is added to a breast pump device. Alternatively, one or more components of a breast pump device may be altered or adjusted to cause vibrations. In other embodiments, a separate device may be used to generate vibrations. Examples of these types of embodiments include but are not limited to modulating the vacuum pump of a breast pump device, modulating the solenoid of a breast pump device, or adding a vibratory motor, a piezoelectric element, a speaker, a shaking element on the bottom of the pump motor housing or pump, an off-center rotary weight on the motor or shaft, or teeth in the wall of the piston housing that allow the diaphragm to “chatter” forward and backward. The vibration source can be built into the pump, the flange or an external device.

In some example, an example vacuum motor device for facilitating milk extraction from a breast of a user can include: a first chamber; a second chamber coupled to the first chamber; and a voice coil motor configured to cause air to flow into the second chamber and out of the first chamber during a breast pumping cycle to create suction for extracting the milk.

The ideal breast pump should be hands-free, giving the mother freedom to multitask while pumping. For a compact wearable breast pump, the challenge is fitting the vacuum motor, solenoid and associated electronics within the compact housing. Ideally, the vacuum motor can not only create the vacuum, but also release the vacuum, thus removing the need for a separate solenoid and saving space. To date, rotary diaphragm DC motors and piezo electric crystal based motors have been contemplated for these compact wearable breast pumps, but both technologies can create limitations in performance or discretion.

In a typical breast pump system, a DC electric motor generates vacuum by accumulation of negative pressure within a system, generally multi-chambered system. In the same typical system, the release of this vacuum is accomplished by the activity of a solenoid. The power to both elements is provided by AC or DC power from the wall socket port or from a battery or from a AC to DC power converter power supply.

In a vibration wave form breast pump, additionally the system would include a leak hole, secondary actuator element to open a vent, secondary actuator system to create a pinch or backpressure by partially closing the tubing periodically, or by an external shaking element as described. In embodiments described herein, several novel examples of alternative configurations not contemplated previously include use of a vacuum motor configured to release vacuum present in a first chamber and also generate a custom vibration waveform through the motor's actuation. In additional embodiments not previously contemplated the vacuum motor could include a voice coil actuator which enables the pump system to comprise one or more of three actions of a typical breast pump multi-component system in a single component system.

For example, in a typical breast pump system, a vacuum motor accumulates negative pressure in the vacuum chamber and then the release of this pressure is accomplished by a solenoid. In a voice coil actuator system, the position of the diaphragm or solid element that is moving to create vacuum accumulation with one or more valve systems could also be configured to change position such that a vent would be opened to enable the depressurization of the system at desired time intervals. In alternative configurations of a voice coil actuator system, the diaphragm or solid moving component within the voice coil system could be configured to activate one or more different flow channels which include but are not limited to the option to accumulate vacuum, release pressure partially or fully, or slightly release pressure on an oscillatory or partially oscillatory time sequence such that vibration through leakage could occur, or slightly impinge a back pressure on an oscillatory or partially oscillatory time sequence such that vibration through backpressure could occur.

### BRIEF DESCRIPTION OF THE DRAWINGS

FIG. 1 is a perspective view of a currently available electric breast pump system:

FIG. 2 is a time versus pressure diagram, showing a vibration applied via a breast pump by modulating a suction waveform along part of the suction induction breast pump curve, according to one embodiment:

FIG. 3 is a time versus pressure diagram, showing a vibration applied via a breast pump by modulating a suction waveform along all of the suction induction breast pump curve, according to an alternative embodiment;

FIG. 4 is a time versus pressure diagram, showing a vibration applied via a breast pump by modulating a suction waveform along the suction induction breast pump curve, except for a resting hold state at the lowest point in the vacuum, with no vibration or oscillation effect, according to one embodiment;

FIG. 5 is a time versus pressure diagram, showing a breast pump suction curve with a stair-step vibratory stimulation pattern of short stair-step bursts, according to one embodiment:

FIG. 6 is a time versus pressure diagram, showing a breast pump suction curve with alternating and/or independently modulated wave cycles, one including an oscillating effect and another including no oscillating effect, according to one embodiment;

FIG. 7 is a time versus pressure diagram, showing a breast pump suction curve including a drop-in pressure, a stair-step increase in pressure, and an additional cycle, with vibratory effects on at least part of the waveform curve segments, according to one embodiment:

FIGS. 8A-8D are time versus pressure diagrams that depict exemplary vibratory waveforms, each of which includes a vibration segment and a smooth segment during parts of the wave rise, fall, and/or hold segment(s), according to one embodiment:

FIG. 9 depicts a time versus pressure curve and exemplary motor and solenoid control signal curves, illustrating a modulating effect of the control signals on an oscillating pressure reduction curve from a breast pump suction waveform, according to one embodiment;

FIG. 10A is a graph showing a vacuum waveform with a stair-step vibration pattern, according to one embodiment;

FIG. 10B is a graph showing a vacuum waveform with an oscillating increase and decrease vibration pattern, according to an alternative embodiment:

FIG. 11 illustrates a PCB, a pump motor, and a solenoid of a breast pump device, of which one or more may be used to drive activity of the breast pump waveform and waveform effects, according to various embodiments:

FIG. 12 is a side view of a breast pump flange and receptacle, with a vibration motor coupled with the breast pump flange, according to one embodiment:

FIG. 13 is a perspective view of a breast pump system including abreast pump flange and a separate vibration motor designed to be held by the user to mechanically vibrate the breast, according to one embodiment;

FIG. 14 is a side view of a breast pump flange with a moving membrane and an eccentric motor, according to one embodiment;

FIG. 15 is a schematic view of a breast pump device according to another embodiment.

FIG. 16 is a schematic view of the breast pump device of FIG. 15 with the piston in a first position.

FIG. 17 is a schematic view of the breast pump device of FIG. 15 with the piston in a second position.

FIG. 18 is a schematic view of the breast pump device of FIG. 15 with the piston in a third position.

FIG. 19 is a schematic view of the breast pump device of FIG. 16 with the piston in a fourth position.

FIG. 20 is a schematic view of another example breast pump device.

FIG. 21 is a schematic view of the breast pump device of FIG. 20 within the interior space of a funnel.

FIG. 22 is an example voice coil with a Halbach array for use in a breast pump device.

FIGS. 23A and 23B depict another example breast pump device operating in an example vacuum mode.

FIGS. 24A and 24B depict the example breast pump device of FIGS. 23A and 23B, operating in an example vibration mode.

FIGS. 25A and 25B depict the example breast pump device of FIGS. 23A and 23B, operating in an example release mode.

FIG. 26 is another example breast pump device, utilizing a voice coil actuator and a solenoid.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

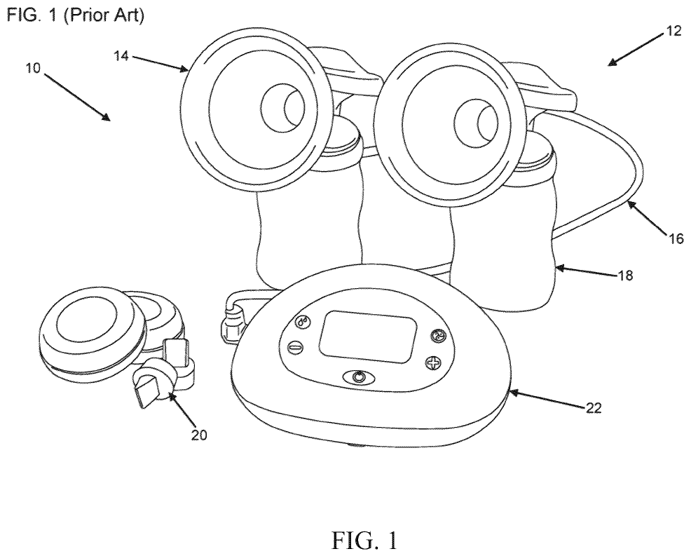

Referring to FIG. 1, one example of a currently available electric breast pump system 10 is shown. In this example, the system 10 includes a breast contacting portion 12 and a control unit 22. The breast contacting portion 12 typically includes two funnels 14 (or “shields”) for directly contacting and fitting partially over a woman's breasts, two milk collection receptacles 18 connected to the funnels 14, two duckbill valves 20 (or “membranes”) that reside inside the breast contacting portion 12 when in use, and a tube connector 16 for connecting the funnels 14 with the control unit 22. The control unit 22 typically includes several primary components, all of which are inside the housing of the control unit 22 and thus not visible in FIG. 1. For example, the control unit 22 typically houses a vacuum motor for generating vacuum (or “suction”) force that is conveyed through the tube connector 16 to the funnels 14, a solenoid that helps release vacuum pressure from the system 10, and electronics for driving the system 10.

Different terminology is sometimes used by people of skill in the art to refer to the various parts of a breast pump system 10. For example, the breast contacting portion may be referred to as a “milk extraction set” or a “disposable portion,” the funnels 14 are often referred to as “breast shields,” and the control unit is sometimes simply referred to as “the pump.” This application will typically use terminology as described immediately above, but these terms may in some cases be synonymous with other terms commonly used in the art. Therefore, the choice of terminology used to describe known components of a breast pump system or device should not be interpreted as limiting the scope of the invention as defined by the claims.

As mentioned in the Background section, currently available electric breast pump systems, such as the system 10 of FIG. 1, operate by applying vacuum force to the breast and releasing the vacuum force repeatedly during a pumping session. Each application and release of vacuum is referred to herein as one “cycle,” where each cycle begins as vacuum force starts to be applied and ends right before vacuum starts to be applied again. The pattern created on a graph of pressure versus time by an operating breast pump may be referred to herein as a “pumping waveform.”

Currently available breast pumps do not vibrate or generate vibrations in the breast as part of their regular function. Instead, they provide smooth, vibration-free suction and release cycles. In general, the methods described herein use one or more mechanisms to add vibrations to at least part of the breast pump cycle, in order to enhance the function of the breast pump and thus facilitate milk extraction from the breast. The application sometimes refers to the pumping waveform with the addition of vibrations as a “vibration waveform.” In other words, the “vibration waveform” may refer to any breast pumping waveform that has vibrations added to it.

Current breast pumps allow for changing the cycle speed and the suction pressure of the pump. The Hagen-Poiseuille fluid dynamic equation, derived from the approximation of a Newtonian fluid undergoing laminar flow, reads as follows: ΔP=(8μLQ)/(πR4), where ΔP=pressure difference (in the milk duct), L is length (of the milk duct), μ=dynamic viscosity (of the milk), Q=volumetric flow rate, and R=radius (of the milk duct). Current pumps only target ΔP by adjusting the suction pressure. Milk and colostrum can be approximated as a Newtonian fluid, and the dimension of the radius of the milk duct pipes can also enable us to be reasonably certain that almost all flow regimes encountered would consist of laminar flow segments. As a result, a Hagen-Poiseuille derivation from the shear stress equation τ=−μ*(dv/dr), where μ=viscosity, v=velocity of the fluid, and r=the position along the radius in the tube, should represent a reasonable approximation. As such, the cycle speed of a breast pump affects how many suction and release cycles the breast pump operates in a minute but does not affect the volume flow rate during a cycle.

The devices, systems and methods described in this application enhance breast pump function by applying vibrations to reduce shear stress z along a given radius of breast milk duct (or “conduit”), so that more volume in the duct will move at a higher velocity. The applied vibrations increase Q (volumetric flow rate) when other parameters are fixed, and they may also stimulate the breast to induce let down and further increase the radial dimension R of the breast milk duct along critical flow restriction points. The decrease in μ from vibration may also be explained by the following equation, F=μA(υ/y). With vibration, the friction between the fluid and the walls of the duct is decreased, thereby reducing the amount of force needed to maintain the flow velocity. In addition, or as a separate effect, vibration may stimulate let down, which increases the cross-sectional area of each milk duct. Going back to the Hagen-Poiseuille fluid dynamic equation, given a fixed ΔP, μ must decrease and Q must increase to balance the equation. Let down induces an increased radius and corresponding increase in Q, assuming the same pressure gradient.

The devices, systems and methods described herein use oscillation vibration patterns to induce increased milk flow from the breast during pumping, through one or more mechanical pathways. In various examples and embodiments, the devices, systems and methods may produce vibrations (or the vibratory waveform) with any suitable pattern, sin, shape, timing, etc. For example, in any given embodiment, the frequency of the vibrations or oscillations may range from as low as just above 0 Hz to as high as 10 MHz. There may be an ideal frequency range of the vibrations for comfort and the ability of the woman to feel the vibrations, which may for example be in a range of about 5 Hz to about 10 Hi. Alternatively, a wider range of about 2 Hz to about 20 Hz may be ideal in some embodiments. Generally, if the vibration frequency is too high, the woman will not feel the vibrations. On the other hand, high frequency vibration in the ultrasound range might be helpful in some instances, such as for unclogging milk ducts and alleviation of mastitis.

Just as any suitable type of vibrations may be applied, according to various embodiments, any suitable devices may be used to produce the vibrations, examples of which are described below. Therefore, this application should not be interpreted as being limited to any particular type or pattern of vibrations or any particular device for inducing vibrations.

As just mentioned, this application describes devices, systems and methods that help enhance breast milk pumping by vibrating the milk ducts to increase the volumetric flow rate of the milk. A typical breast pump includes a vacuum motor and a solenoid. During each pumping cycle, the vacuum motor turns on, creating pressure at the breast and thus helping express milk. At the end of the cycle, the pressure is released by turning on the solenoid to normalize the pressure in the breast pump flange. The cycle is then repeated. By “repeated,” it is meant simply that multiple cycles run in succession, for as long as the breast pump is activated. In some cases, the same cycle may be repeated over and over again—i.e., cycles with the same waveform. In other embodiments, the cycles may differ. For example, two different cycles may alternate. Or the cycle waveform may change over time. Or the cycle waveform may be adjustable or have automatic changes overtime, according to a built-in algorithm. Therefore, in any given embodiment, the cycles may repeat or vary over time.

In some embodiments, the vibratory waveform may be generated mechanically by the design of the vacuum pump. For example, in a multiple n-piston-based vacuum pump, m pistons (where m<n) can be non-connected or connected to a release valve, which will create the stepwise vibratory pressure profile. In the multiple n-piston-based vacuum pump, the pistons may be aligned asymmetrically, to provide the vibratory waveform. Alternatively or additionally, valves within the piston vacuum pump may be purposely designed to be “leaky,” to provide a partial release in vacuum to create a more pronounced vibration effect. Other mechanical alterations may include designing a release valve that automatically turns on and off rapidly to create the vibration. The vibration may also be created by a motor squeezing and releasing a bulb or balloon that is in-line with the vacuum pump.

The frequency and amplitude of the generated vibrations may be varied, in order to induce or sustain let down, make let down happen easier by lowering the sensation threshold of the body, and/or vibrate the milk to make it flow more easily by reducing the shear stress of the fluid and/or frictional coefficients of the fluid against the ducts. To conserve battery power, generated vibrations may have a low frequency and a low amplitude. Alternatively, any combination of frequency and amplitude may be used.

Any features or components described in this application for generating a vibratory waveform in a breast pump may be used with or incorporated into any suitable powered or non-powered breast pump device. The vibratory waveform may be used as a third method for controlling the pumping apparatus, in addition to (or as an alternative to) adjusting the breast pump's cycle speed and/or suction level. In various embodiments, the vibratory waveform may be tuned by the user and/or by a feedback control mechanism built into the device. The vibratory waveform may help vary the vibration level within the waveform or against the breast tissue so that the variables of suction, vacuum and vibration could be independently controlled by the user manually or by an automated or adaptive learning computer algorithm, to support the optimization of milk output.

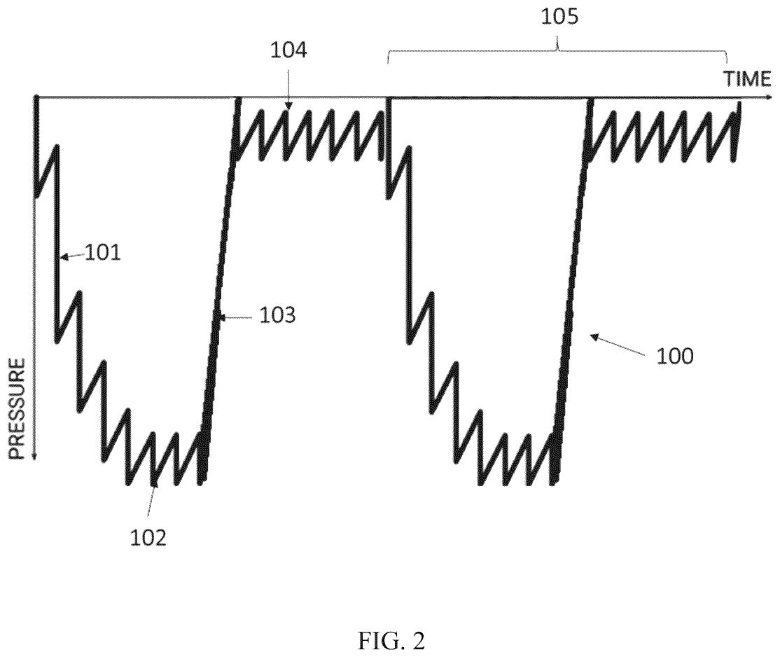

Referring now to FIGS. 2-10B, according to various examples and embodiments, many different vibratory waveform shapes, types, patterns, sizes, etc. may be generated and used in a breast pump device to enhance milk extraction from a breast. FIGS. 2-10B illustrate examples of such vibratory waveforms. Later figures depict examples of devices that may be used to generate the vibratory waveforms. In general, any vibration inducing device described herein may be used to generate vibrations having any waveform or other characteristics, unless specifically described otherwise. Thus, the scope of the present application should not be limited to the use of any specific vibration device or any specific vibratory waveform.

FIG. 2 is a time versus pressure graph that shows one embodiment of a vibratory waveform 100, which may be generated in a breast pump using the methods and devices described herein. Each complete cycle 105 of the vibratory waveform 100 includes an increasing vacuum segment 101 (or “reduction in pressure segment”), a vacuum hold segment 102, a vacuum release segment 103 (or “normalizing the pressure segment” or “venting segment”), and a final hold segment 104 (or “normalized pressure hold segment”). In this embodiment, the vibrations of the vibratory waveform 100 are applied during the vacuum segment 101, the vacuum hold segment 102, and the final hold segment 104, but not during the vacuum release segment 103. The oscillatory effect of the normalized pressure hold segment 104 may occur at the normalized pressure, slightly higher than normalized pressure, or most preferably lower than normalized pressure—e.g., a slight vacuum, to help maintain the breast in the correct suction position within the flange of the breast pump. The waveform 100 may be repeated for any number of cycles 105, in the same pattern or a different pattern. The pattern of the waveform 100 may be changed, according to various embodiments, automatically, manually or both. For example, the pattern may be adjusted manually by the user by varying settings of the breast pump device. Alternatively or additionally, the pattern may be adjusted automatically by a control unit of the breast pump device, which may be directed via computer software through tunable or reactive learning interactions.

As mentioned above, currently available breast pump systems typically allow a user to adjust (or adjust automatically) the cycle speed and suction pressure of the system. Referring to the waveform 100 of FIG. 2, adjusting the cycle speed would change the “width” of each cycle 105 along the horizontal “time” axis of the graph. A faster cycle speed equates to higher frequency, and a lower cycle speed to lower frequency. Adjusting the suction pressure would change the “height” or “depth” of the curve along the vertical “pressure” axis of the graph. According to various embodiments described herein, the user and/or the control unit of the breast pump system may adjust vibrations in addition to or as an alternative to adjusting cycle speed and/or suction pressure. Vibration adjustments may include, for example, turning vibrations on or off, making vibrations occur over different portions of the waveform 100, and/or changing a pattern or depth/strength of each vibration. In some embodiments, for example, the breast pump system may include one or more dials, switches, buttons, sliders or the like, for making the adjustments. Some embodiments may include a separate controller, such as a remote control unit or a computer application downloaded on a smart phone, tablet, etc. Generally speaking, any given embodiment may allow a user to adjust or control vibrations, cycle speed and/or suction pressure in any suitable combination.

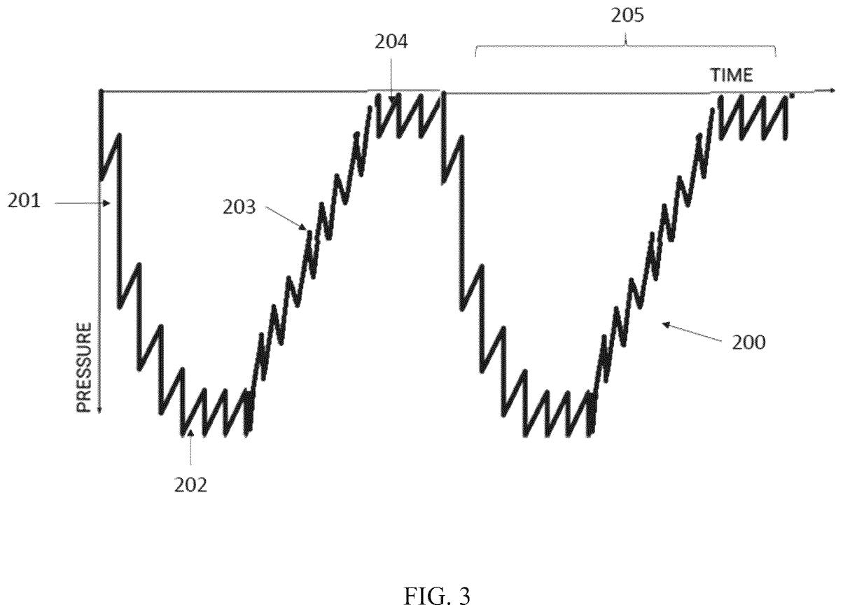

Referring now to FIG. 3, another embodiment of a vibratory waveform 200 for use with abreast pump device is illustrated. In this embodiment, the waveform 200 includes an increasing vacuum segment 201, a vacuum hold segment 202, a slow vacuum release segment 203, and a restart segment 204 at or near normalized pressure, which may contain a vibratory pattern. In this embodiment, vibrations are applied throughout the entire cycle 205 of the waveform 200, although vibrations during the restart segment 204 are optional. According to various embodiments, the segments 201, 202, 203, 204 may repeat in any configuration of these patterns or other patterns of vibration, suction, stair step, etc. The vibration patterns disclosed herein are also be interchangeable between each other, so that a user of a breast pump device may experience multiple different types of patterns within one operational period of the device.

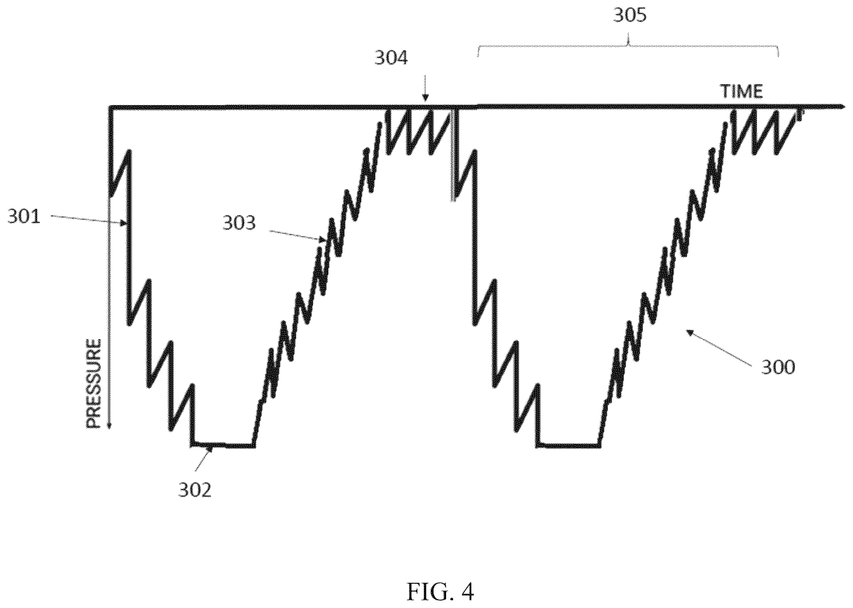

FIG. 4 shows another embodiment of a vibratory waveform 300 for use with abreast pump. In this embodiment, each cycle 305 of the waveform 300 includes an increasing vacuum segment 301, ahold vacuum segment 302, a slow vibratory vacuum release segment 303, and a near normalized pressure segment 304. In this embodiment, vibrations are applied during all segments other than the hold vacuum segment 302, which is vibration free. For this waveform 300, the normalize pressure segment 304 is optional, meaning that in some embodiments one cycle 305 may end with the vibratory vacuum release segment 303, and the next cycle may immediately begin with the increasing vacuum segment 301.

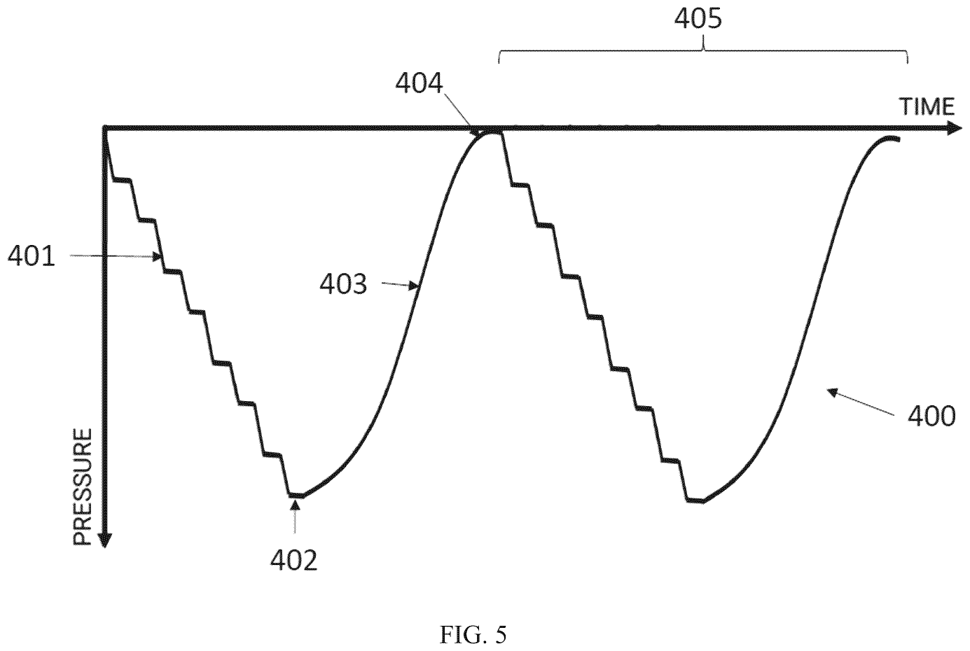

FIG. 5 depicts another embodiment of a vibratory waveform 400 for a breast pump suction profile. In this embodiment, each cycle 405 of the waveform 400 includes a vacuum segment 401, a maximum vacuum segment 402, a vacuum release segment 403, and an end cycle segment 404. The vacuum segment 401 has a stair-step pattern of vibrations applied to it. The maximum vacuum segment 402 may include a hold period, during which vacuum is maintained, but such a period is optional.

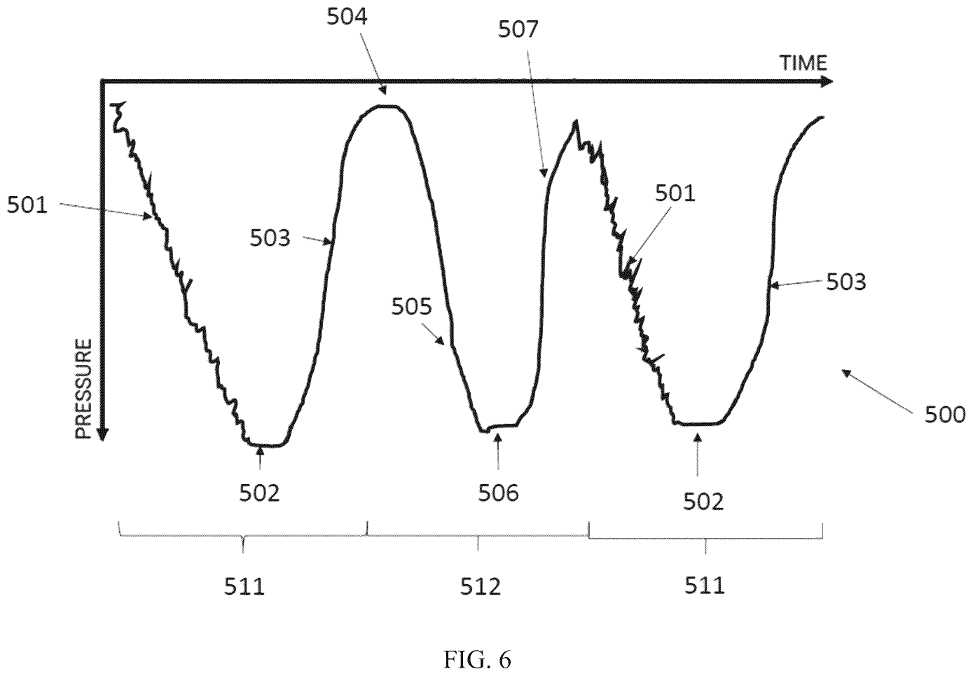

With reference now to FIG. 6, another embodiment of a vibratory waveform 500 for a breast pump is illustrated. This embodiment includes two types of waveform cycles—a first cycle type 511 and a second cycle type 512. The first cycle type 511 includes an increasing vacuum segment 501 with micro-oscillation vibrations, and a hold vacuum segment 502, a vacuum release segment 503 and an end segment 504, all with no vibrations. The second cycle type 512 includes an increasing vacuum segment 505, a hold vacuum segment 506, and a vacuum release segment 507, all with no vibrations. These cycles 511, 512 of the vibratory waveform 500 may be performed in any order desired by a user. The embodiment of FIG. 6 includes two different types of cycles 511, 512 in a single waveform 500, but other embodiments may include more than two different types of cycles, different patterns of differing cycles, oscillation between two or more cycle profiles, and/or the like. In various embodiments, any of the waveform shapes, patterns, types and/or sizes described herein may be combined with any other waveform shapes, patterns, types and/or sizes, whether described herein or not, in any combination and number, without departing from the scope of this disclosure.

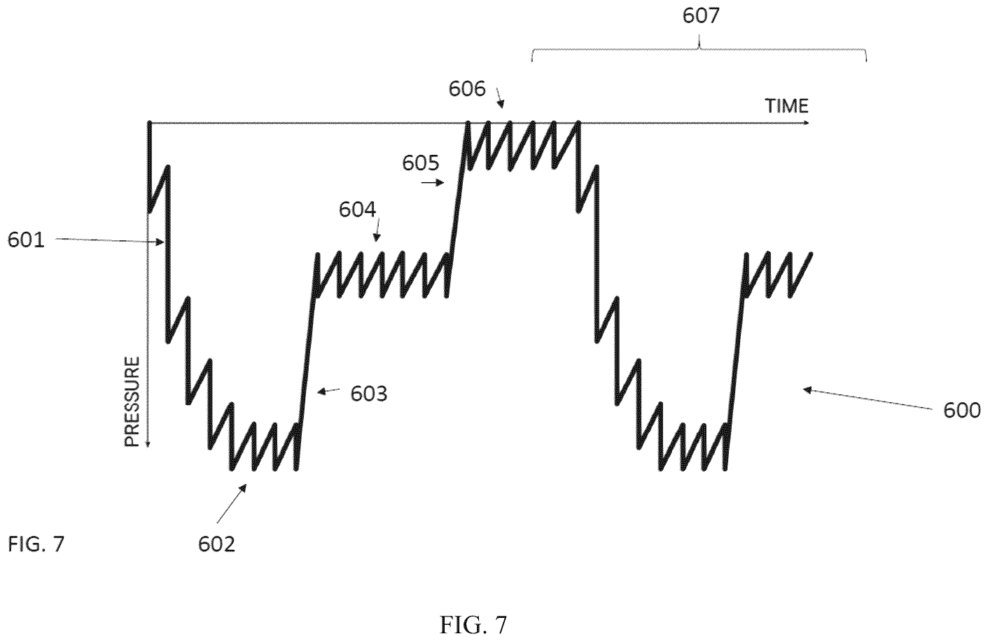

FIG. 7 depicts another embodiment of a vibratory waveform 600 for a breast pump suction curve, in which each cycle 607 includes a vacuum increase segment 601, a vacuum hold segment 602, a first vacuum release or vent segment 603, a partial reduced vacuum hold segment 604, a second vacuum release or vent segment 605, and an end of cycle segment 606, at which pressure is near ambient normal. Vibrations are applied at all segments other than the first vacuum release segment 603 and the second vacuum release segment 605. Variations on this embodiment of the waveform 600 may include different combinations of more or fewer hold segments, vacuum increase segments and/or vacuum decrease segments. Additionally, the same elongated stair-step vibration pattern used in the vacuum increase segment 601 may be applied in one or both of the vacuum release segments 603, 605, in alternative embodiments, to more slowly reduce the vacuum to one or more limits, to facilitate the stimulation of let down and/or the stimulation or production of breast milk and/or colostrum.

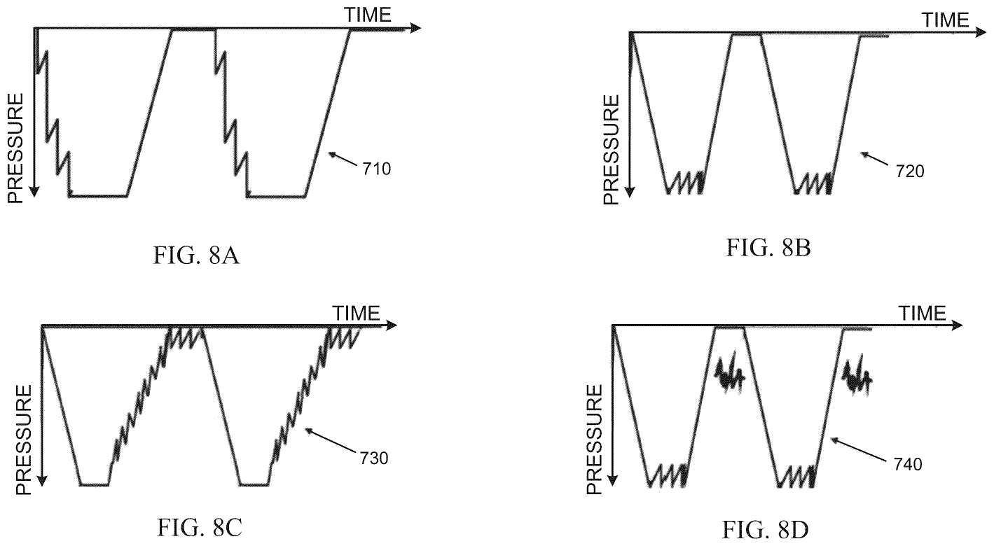

FIGS. SA-8D show four different embodiments of breast pump suction waveform profiles with varying segments of oscillation and/or vibratory effects. FIG. 8A shows a waveform 710 with a vibration effect on the increase in vacuum side of the cycle. FIG. 8B shows a waveform 720 with a vibration effect within the maximum vacuum segment. FIG. 8C shows a waveform 730 with a vibration effect during and immediately after venting to a near normalized pressure segment. FIG. 8D shows a waveform 740 with a vibration effect upon venting to a near normalized pressure segment including increasing the pressure slightly above the current atmospheric pressure in which the pump is operating if desired. These effects may be controlled by a micro-processor within the control unit of the breast pump device (or separate from the breast pump device), which can tune the effects of one or more motors and/or one or more solenoids to adjust the effect over different segments of the breast pump to produce the desired effect while pumping the breast.

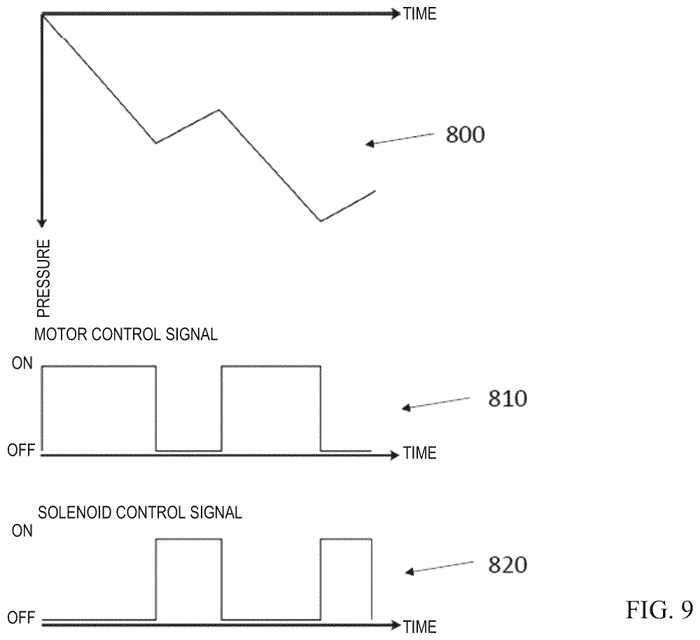

FIG. 9 includes a time versus pressure curve 800 in parallel with a motor control signal on/off curve 810 and a solenoid control signal on/off curve 820. In various embodiments, the motor and/or the solenoid of a breast pump may be tuned/adjusted by a user to produce the desired vibration and vacuum waveform 800 for pumping. This effect and/or the action of the motor(s) and/or solenoid(s) to create the vibrations and/or controlled waveform effect may additionally or alternatively be adjusted by a control unit of the breast pump, programmed with software, to facilitate specific wave forms at different times, as desired by the user and/or as informed to the control unit by sensors or feedback from the user.

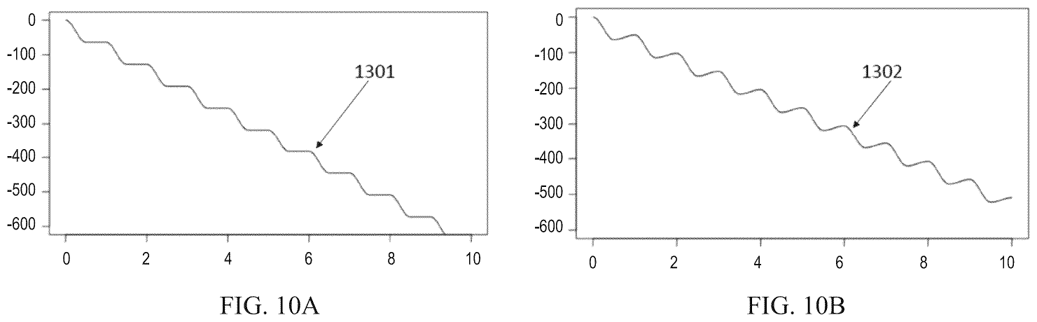

FIGS. 10A and 10B are graphs illustrating two different embodiments of vibratory waveforms. In FIG. 10A, the vibratory waveform 1301 has a stair-step pattern. One method for generating such a pattern is to rapidly turn the breast pump on and off repeatedly. This may be achieved, for example, by using a stepper motor or a DC motor. When the breast pump is on, vacuum is increased. When the pump is off, vacuum is held.

In FIG. 10B, the vibratory waveform 1302 has a wavy pattern created by repeated oscillatory increases and decreases in vacuum. One method for generating this type of wavy patterned waveform is by having a separate vacuum motor or m piston (where m≥1 and m≤n) within a n-piston vacuum motor increase and/or decrease the vacuum within the system. Another method to generate this pattern is a controlled partial release of vacuum by using a solenoid.

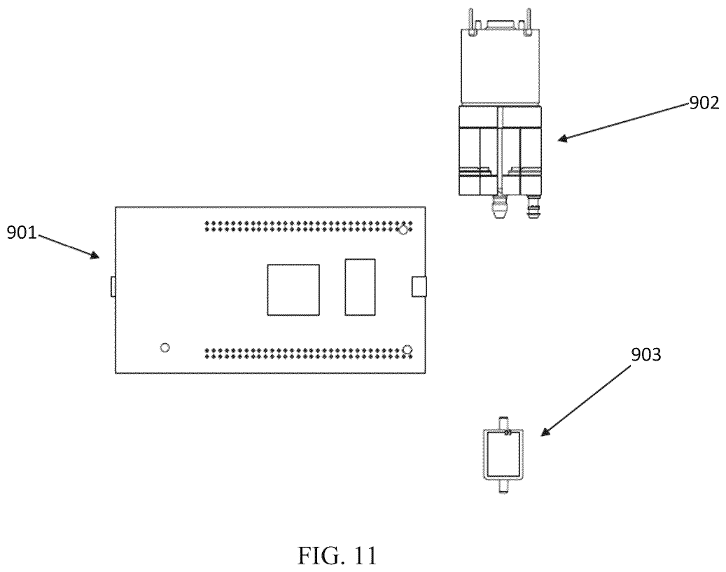

FIG. 11 illustrates three components that may be included in a breast pump device or system and that may be used, in various combinations, to provide a vibratory waveform. These components may include a printed circuit board (PCB) 901 (or other similar electronic components), a motor 902, and a solenoid 903. Various embodiments of a breast pump may include multiple PCBs 901, multiple motors 902, and/or multiple solenoids 903, and that fact will not be repeated each time any of these components is mentioned. The PCB 901 may work together with the motor 902 and/or the solenoid 903 to provide vibrations to the breast pump cycle, as described above. In alternative embodiments, other types of pressure venting devices may be passively, electrically, or mechanically actuated in combination with the pump motors, pressure regulator valves, and/or other components, to create the desired wave form within the suction induction curve.

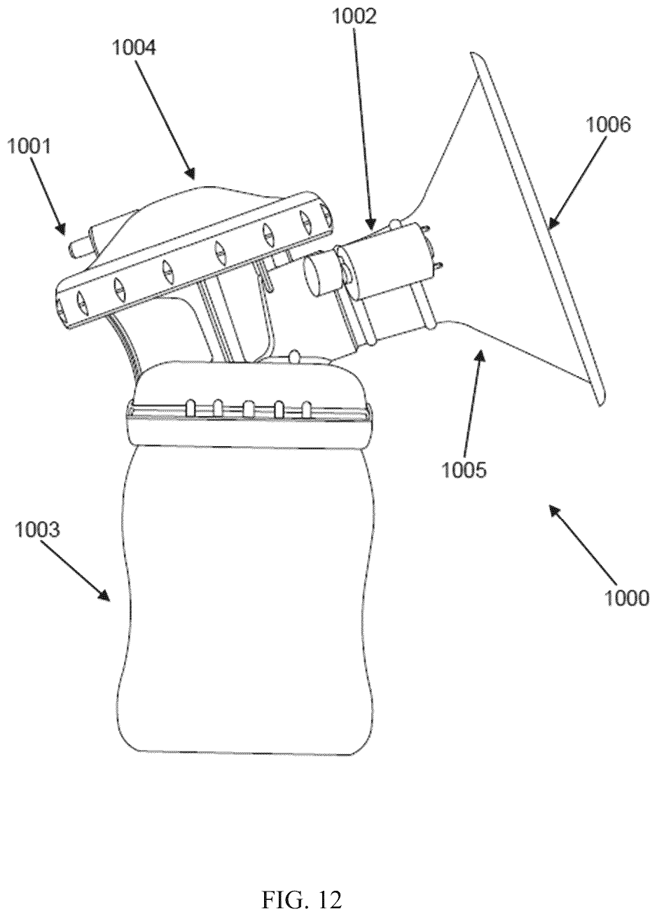

FIG. 12 is a side view of a breast pump device 1000, according to one embodiment. This and several following figures will refer to the breast contacting portion of the breast pump system as the “breast pump device.” Not shown are the control unit (or “pump”) and the tubing for connecting the breast pump device with the control unit. As mentioned previously, the specific terminology used for various components of a breast pump system should not be interpreted as limiting.

In this embodiment, the breast pump device 1000 includes a vacuum port 1001, a pressure regulation diaphragm 1004, a collection receptacle 1003 for milk or colostrum, a vibration device 1002, and a funnel 1005 with an opening 1006 for accepting a breast. The vibration device 1002 is a small vibration inducing motor attached to a proximal portion of the funnel 1005. In alternative embodiments, the vibration device 1002 may be attached to a different part of the breast pump device 1000, such as but not limited to a flange, the collection receptacle 1003 or the diaphragm 1004. In the pictured embodiment, the vibration device 1002 directly vibrates the funnel 1005, which conducts the vibrations into the breast tissue received in the opening 1006. The vibration device 1002 may generate any of the various types and patterns of vibratory waveforms described above or any other suitable vibrations.

FIG. 12 is a side view of a breast pump device 1000, according to one embodiment. This and several following figures will refer to the breast contacting portion of the breast pump system as the “breast pump device.” Not shown are the control unit (or “pump”) and the tubing for connecting the breast pump device with the control unit. As mentioned previously, the specific terminology used for various components of a breast pump system should not be interpreted as limiting.

In this embodiment, the breast pump device 1000 includes a vacuum port 1001, a pressure regulation diaphragm 1004, a collection receptacle 1003 for milk or colostrum, a vibration device 1002, and a funnel 1005 with an opening 1006 for accepting a breast. The vibration device 1002 is a small vibration inducing motor attached to a proximal portion of the funnel 1005. In alternative embodiments, the vibration device 1002 may be attached to a different part of the breast pump device 1000, such as but not limited to a flange, the collection receptacle 1003 or the diaphragm 1004. In the pictured embodiment, the vibration device 1002 directly vibrates the funnel 1005, which conducts the vibrations into the breast tissue received in the opening 1006. The vibration device 1002 may generate any of the various types and patterns of vibratory waveforms described above or any other suitable vibrations.

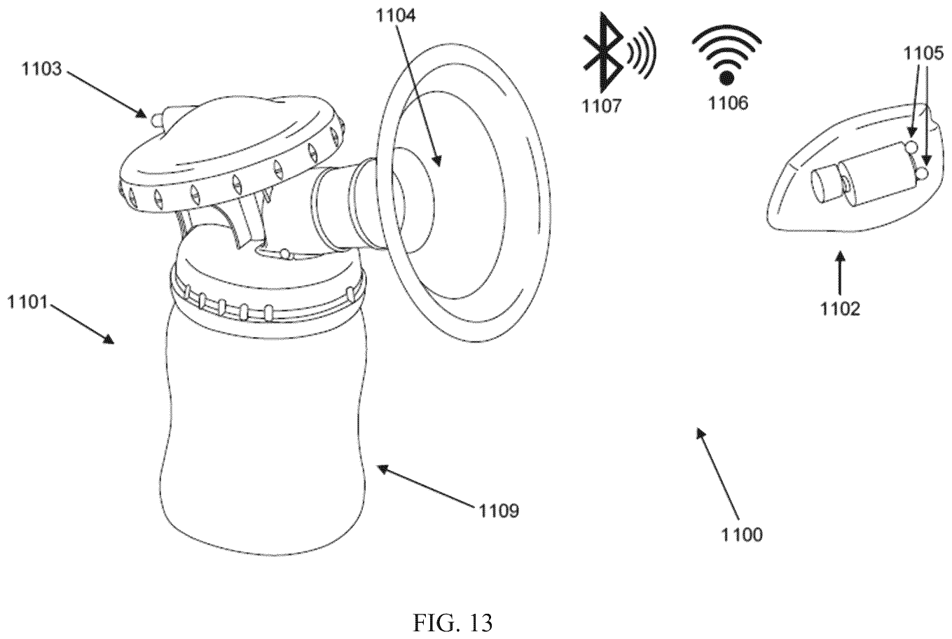

Referring now to FIG. 13, in another embodiment, a breast pump system 1100 may include a breast pump device 1101 and a separate vibration device 1102. Again, the source of suction—i.e., the breast pump housing mechanism with the motor(s), power cord, etc.—is not shown, but it may be included as part of the system 1100. The breast pump device 1101 includes a vacuum port 1103, a funnel 1104 and a collection receptacle 110′), among other parts. The separate vibration device 1102 may include a small motor for creating vibrations, and it may be held by the user against the breast or attached (e.g., adhesive) temporarily to the breast. The vibration device 1102 may include one or more signal transmitters 1105, receivers and/or transceivers, which communicate with a breast pump control unit (not shown) through wired or wireless connections, such as WIFI 1106 and/or Bluetooth 1107. Although not required, this communication could, in combination with sensors in the vibration device 1102 and/or the breast pump device 1101, provide feedback for the microcontroller to adjust the actuation of the pressure in the breast pump waveform and/or the level of vibration produced by the vibration device 1102. This feedback loop may be preset into the breast pump system 1100 in some embodiments.

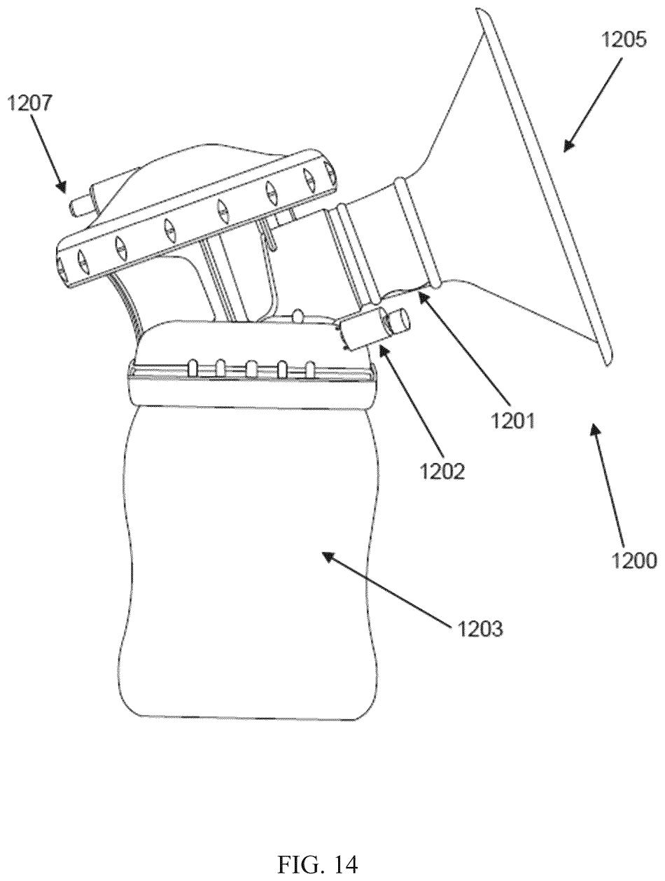

FIG. 14 is a side view of a breast pump device 1200 according to another embodiment. In this embodiment, the device 1200 includes all the features of atypical breast pump device, such as a collection receptacle 1203, a funnel 1205, a suction port 1207, etc. In addition, the device 1200 includes an eccentric motor 1202 attached to the top or lid portion of the collection receptacle 1203. The eccentric motor 1202 generates vibrations, which vibrate a membrane 1201 disposed in the funnel 1205, thus resulting in an oscillatory increase and decrease of vacuum (vibration) in the vacuum waveform. The eccentric motor 1202 may communicate to the breast pump control unit through wireless or wired technologies. The eccentric motor 1202 may be attached as part of the breast pump device 120 or may be a separate piece that can be attached by the user, according to various embodiments.

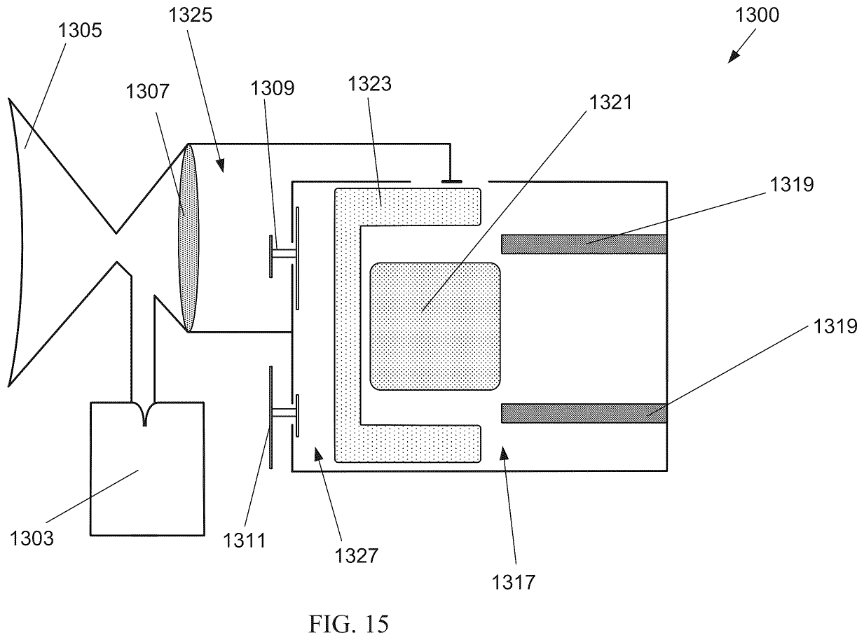

FIG. 15 is a schematic view of a breast pump device 1300 according to another embodiment. In this embodiment, the device breast pump device 1300 includes some of the features of a typical breast pump device, such as a collection receptacle 1303 and a funnel 1305. In addition, the device includes a diaphragm 1307, a plurality of valves 1309, 1311, a first chamber 1325, a second chamber 1327, and a drive system 1317.

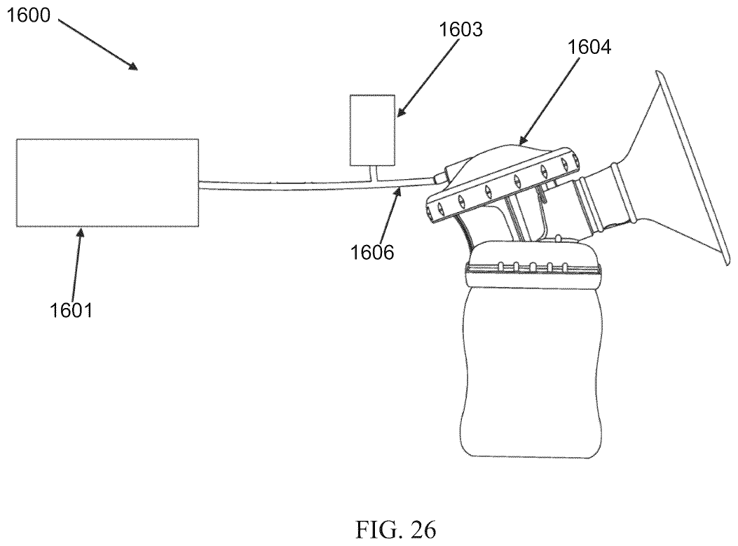

In general, the breast pump device 1300 can function to provide one or more of: (i) vacuum generation to suction breastmilk; (ii) venting to release the suction; and (iii) vibration to assist in the expression of the breastmilk. In some examples, the drive system 1317 performs one, two, or all three of these functions. In other examples, the drive system 1317 performs one or two of the functions, and another drive system performs the other function(s). See, for example, FIG. 26 below.

More specifically, the drive system 1317 operates to create a vacuum within the first chamber 1325. The vacuum causes a deviation in the position in the diaphragm 1307, which creates a vacuum within the funnel 1305 when the opening of the funnel 1305 is placed on and is covered by a woman's breast. The vacuum creates a suction effect against the breast, which causes the woman to express breastmilk into the funnel 1305. The expressed breastmilk then travels through the funnel 1305 into the collection receptacle 1303.

In alternative embodiments, the diaphragm is not used. This forms an open system where the suction from the vacuum is provided directly to the woman's breast. Other configurations are possible.

In the example of FIG. 15, the drive system 1317 is a voice coil actuator drive system 1317. In the examples shown, the voice coil actuator drive system 1317 is a direct-drive linear motor. Generally, current flowing through the coil of the actuator interacts with a permanent magnetic field of a piston and generates a force vector perpendicular to the direction of current.

While the examples provided herein include voice coil actuators, other motor or drive systems could be used in addition to a voice coil actuator. Other drive systems that could be used would include, but not be limited to, a rotary piston motor, DC Shunt Motor, Separately Excited Motor, DC Series Motor, PMDC Motor, Piezo motor, DC Compound Motor, AC motor, Synchronous Motor, Induction Motor, Stepper Motor, and many other types of systems such that they could conduct air in or out of a system.

The voice coil actuator drive system 1317 includes a coil 1319, a magnetic portion 1321, and a piston 1323. In some examples, the coil 1319 is supplied with an electrical current from a power source. The current flowing through the coil 1319 creates a magnetic field. The magnetic field interacts with the magnetic portion 1321 and creates a force vector that pushes the magnetic portion 1321 further into the coil 1319 or further out of the coil 1319. The direction of the force vector is reversable by reversing the polarity of the current flowing through the coil 1319.

In some examples, the magnetic portion 1321 is also attached to a piston 1323. The position of the piston 1323 is dependent on the magnetic portion 1321, so as the magnetic portion 1321 is moved by the operation of the voice coil drive system 1317, the piston 1323 is also moved.

As noted above, in some examples, the breast pump device 1300 includes a first chamber 1325 and a second chamber 1327. The first chamber 1325 is adjacent to the diaphragm 1307, so that at a portion of the first chamber 1325 is defined by the diaphragm 1307. The diaphragm 1307 is made from a flexible material and provides a seal between the first chamber 1325 and an exterior space (e.g., ambient environment). Thus, variations in the pressure within the first chamber 1325 result in variations in the shape of the diaphragm 1307. For example, as the pressure within the first chamber 1325 decreases, the diaphragm 1307 deviates into the first chamber 1325. As the pressure within the first chamber 1325 stabilizes, the diaphragm 1307 deviates back towards a center position.

The second chamber 1327 houses the drive system 1317. In some examples, the piston 1323 is sized so that it touches and creates a seal with the walls of the first chamber 1325. The first chamber 1325 also includes at least two directional valves 1309, 1311. The first directional valve 1309 separates the first chamber 1325 from the second chamber 1327. The first directional valve 1309 valve is oriented to permit airflow from the first chamber 1325 into the second chamber 1327, but seals and prevents airflow back from the second chamber 1327 into the first chamber 1325. The second directional valve 1311 separates the first chamber 1325 from the exterior space of the breast pump device 1300. The second directional valve 1311 is oriented so that it permits airflow from the second chamber 1327 into the exterior space but does not permit airflow from the exterior space back into the second chamber 1327.

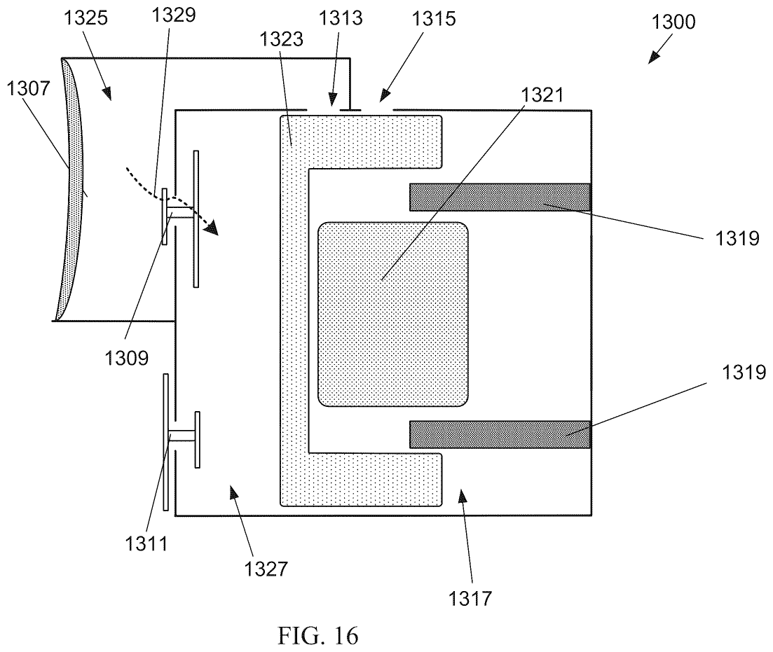

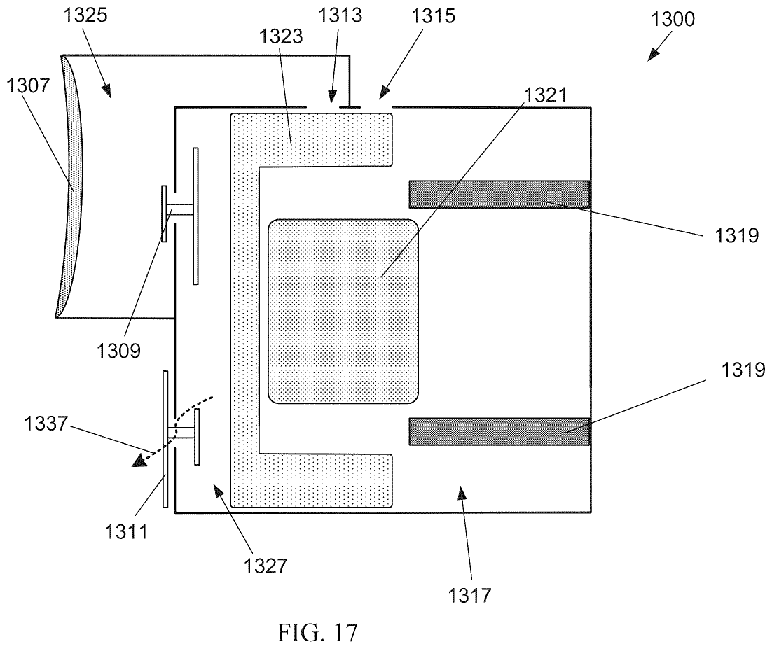

FIGS. 16 and 17 show the breast pump device 1300 of FIG. 15 operating in a vacuum mode. FIG. 16 shows the breast pump device 1300 with the piston 1323 in a first position and FIG. 17 shows the breast pump device 1300 with the piston 1323 in a second position. In the examples of FIGS. 16 and 17, the breast pump device 1300 is illustrated without the funnel 1305 and collection receptacle 1303, however, it should be appreciated that the breast pump device 1300 also may include the funnel 1305 and collection receptacle 1303, as depicted in FIG. 15.

In the example of FIG. 16, the breast pump device 1300 is seen with the piston 1323 in the first position. Comparing FIGS. 16 and 17, while in the first position, the magnetic portion 1321 is positioned further within the coil 1319 than in in the second position, depicted by FIG. 17. As the drive system 1317 operates in vacuum mode and alternates between FIGS. 16 and 17, the piston 1323 moves back and forth between the first position and second position. As the piston 1323 moves from the first to the second position, the piston 1323 creates a positive pressure differential between the second chamber 1327 and the exterior space, so that the pressure within the second chamber 1327 is greater than the pressure than the pressure within the exterior space. This pressure differential forces air out of the second chamber 1327, through the second valve 1311, and into the exterior space, as depicted by the first airflow arrow 1337.

When moving from the second position to the first position, the piston 1323 functions to create a negative pressure differential between the second chamber 1327 and the first chamber 1325, so that the pressure within the second chamber 1327 is less than the pressure within the first chamber 1325. This negative pressure differential results in air being forced from the first chamber 1325, through the first valve 1309, and into the second chamber 1327, as depicted by the second airflow arrow 1329.

As the breast pump device 1300 continues to operate in vacuum mode, the pressure within the first chamber 1325 gradually continues to decrease each time the piston 1323 moves from the second to the first position. The pressure drop in the first chamber 1325 causes a corresponding deflection of the diaphragm 1307 into the first chamber 1325, which creates a negative pressure within the funnel 1305 when affixed to a woman's breast. In some examples, the operation of the breast pump device 1300 in vacuum mode creates a pressure waveform in the funnel 1305 corresponding to the waveform depicted in FIG. 10A.

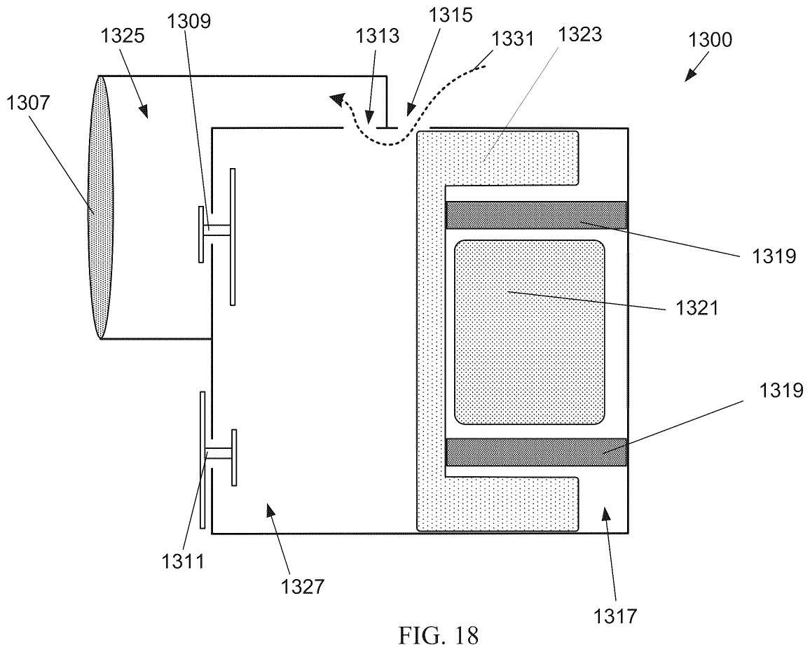

FIG. 18 shows the breast pump device 1300 of FIG. 15 operating in release mode. After the breast pump device 1300 is run in vacuum mode and a negative pressure is created within the funnel 1305, the breast pump device 1300 can be switched to release mode to release the pressure within the funnel 1305. When placed into release mode, the piston 1323 is moved to a third position. In some embodiments, the third position corresponds to a position where the magnetic portion 1321 is received further within the coil 1319 than in the first and second positions.

In some embodiments, the breast pump device 1300 also includes a first port 1313 and a second port 1315. The first port 1313 connects the first chamber 1325 to the second chamber 1327 and the second port 1315 connects the second chamber 1327 to the exterior space. When the piston 1323 is placed in the first and second positions, the first port 1313 and the second port 1315 are generally sealed by the sides of the piston 1323. However, when the piston 1323 is moved back into the third position, the first port 1313 and second port 1315 are both open, so that air can freely travel between the first chamber 1325 and second chamber 1327 and between the exterior space and second chamber 1327.

Due to the pressure differential created between the first and second chambers 1325, 1327 and the exterior space, when the breast pump device 1300 is placed in release mode, air enters the second chamber 1327 from the exterior space and flows into the second chamber 1327, as denoted by airflow arrow 1331. The flow of air from the exterior space into the first chamber 1325 and second chamber 1327 causes the pressure within the first and second chambers 1325, 1327 to neutralize, which also results in the deflection of the diaphragm 1307 back to its steady state position. The deflection of the diaphragm 1307 causes the drop in pressure within the funnel 1305, which results in a loss of suction by the funnel 1305 against the breast. In some examples, the pressure neutralization process that occurs in the release mode creates a pressure waveform within the funnel 1305 that corresponds to the vacuum release segment 403 of FIG. 5, thereby introducing vibratory stimulation into the suction cycle.

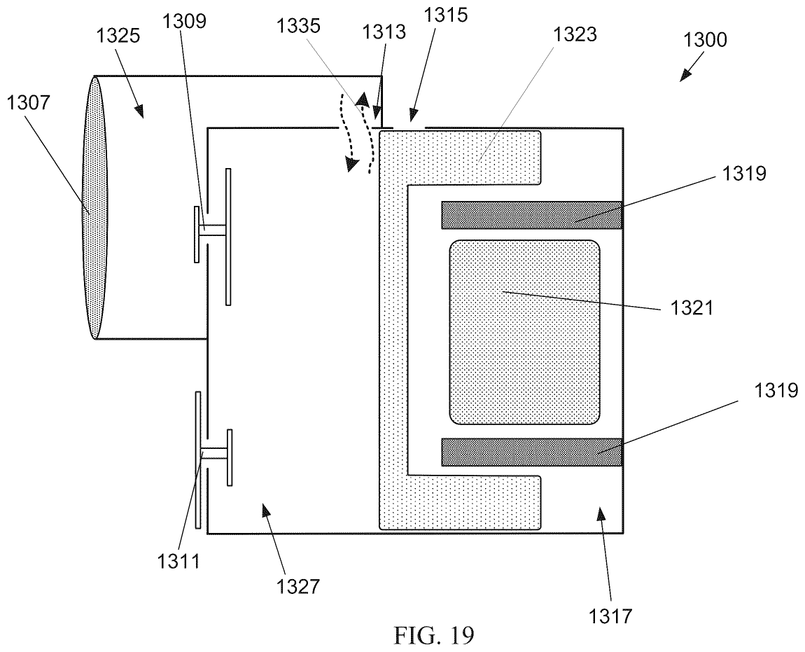

FIG. 19 is a schematic view of the breast pump device 1300 with the piston 1323 placed in a fourth position. In some embodiments, the fourth position is used to produce a vibratory pressure waveform in the funnel 1305. When in the fourth position, the piston 1323 is placed in between the first and third positions. The piston 1323 blocks the second port but does not block the first port.

In vibration mode, the breast pump device 1300 operates by switching between the fourth position, shown in FIG. 18 and the first position, shown in FIG. 16. In some examples, as the piston 1323 moves from the fourth position to the first position, the piston 1323 creates a positive pressure differential between the second chamber 1327 and the first chamber 1325, so that the pressure of the second chamber 1327 is greater than the pressure of the first chamber 1325. This pressure differential forces air from the second chamber 1327 into the first chamber 1325 through the open first port 1313, as shown by the airflow arrow 1335. The influx of air into the first chamber 1325 results in a slight increase in pressure within the first chamber 1325, which corresponds to a deviation in the position of the diaphragm 1307, along with a slight drop in suction within the funnel 1305.

However, in contrast to the release mode, where the pressure within the first and second chambers 1325, 1327 is neutralized with the airflow from the exterior space, in vibration mode, the exterior space remains sealed off from the first and second chambers 1325, 1327. Thus, although the suction in the funnel 1305 drops slightly as the breast pump device 1300 moves from the fourth and first position, the funnel 1305 retains most of its suction. In some examples, when moving from the fourth to first position, the pressure differential between the second chamber 1327 and the exterior space is insufficient to open the second valve 1311, so all of the expelled air from the second chamber 1327 moves through the first port 1313 into the first chamber 1325 rather than through the second valve 1311 into the exterior space. In other examples, some air is pushed through both the first port 1313 and second valve 1311 as the piston 1323 moves from the fourth to the first position.

Next, in some embodiments, the piston 1323 moves back to the fourth position after it reaches the first position. When moving from the first position back to the fourth position, the piston 1323 generates a negative pressure differential between the second and the first chambers 1325, 1327, so that the pressure of the second chamber 1327 is lower than the pressure within the first chamber 1325. This pressure differential causes airflow from the first chamber 1325 into the second chamber 1327 through the first port 1313. This airflow results in a slight decrease in pressure within the first chamber 1325 and deviation of the diaphragm into the first chamber 1325. The movement of the diaphragm 1307 results in a slight increase in suction within the funnel 1305.

By alternating between the first and fourth positions in vibration mode, the breast pump device 1300 creates slight increases and decreases in suction within the funnel 1305, which results in a vibratory effect. In some embodiments, this vibratory effect can help increase the expression of breastmilk from a woman.

In some embodiments, when alternating between the first and fourth positions, the median pressure within the first chamber does not change after successive vibratory cycles. Thus, although slight increases and decreases in suction are provided within the funnel 1305 over successive cycles, the median suction provided over time throughout the successive cycles remains unchanged. This is because air does not pass through the second valve 1311 into or out of the breast pump device 1300. Rather, the air within the breast pump device 1300 stays within the first and second chambers 1325, 1327 and merely alternates back and forth between the first and second chambers 1325, 1327, thereby creating the vibratory effect.

In some embodiments, the breast pump device 1300 can be used to create the vibratory effect while also increasing the suction within the funnel 1305 over successive cycles. In these embodiments, the piston 1323 is operable to move from the fourth position (illustrated in FIG. 19), to the first position (illustrated in FIG. 16), to the second position, (illustrated in FIG. 17), and back to the fourth position (illustrated in FIG. 19).

When moving between the fourth position and the first position, the breast pump device 1300 produces the vibratory effect by pushing air from the second chamber 1327 to the first chamber 1325 through the first port 1313. When moving between the first and second position, the breast pump device 1300 pushes air out of the second chamber 1327 into the exterior space through the second valve 1311. When moving from the second position back to the first position, the breast pump device 1300 creates a negative pressure in the second chamber 1327, pulling air from the first chamber 1325 to the second chamber 1327.

And, when moving from the first position to the fourth position, the piston 1323 of the breast pump device 1300 opens the first port 1313, allowing air to flow from the second chamber 1327 into the first chamber 1325. In some embodiments, for example, the breast pump device 13M) makes multiple cycles between the first and forth positions before making a cycle between the first and second positions. Alternatively, in some examples, the breast pump device 1300 makes multiple cycles between the first and second positions before making a cycle through the first and fourth positions.

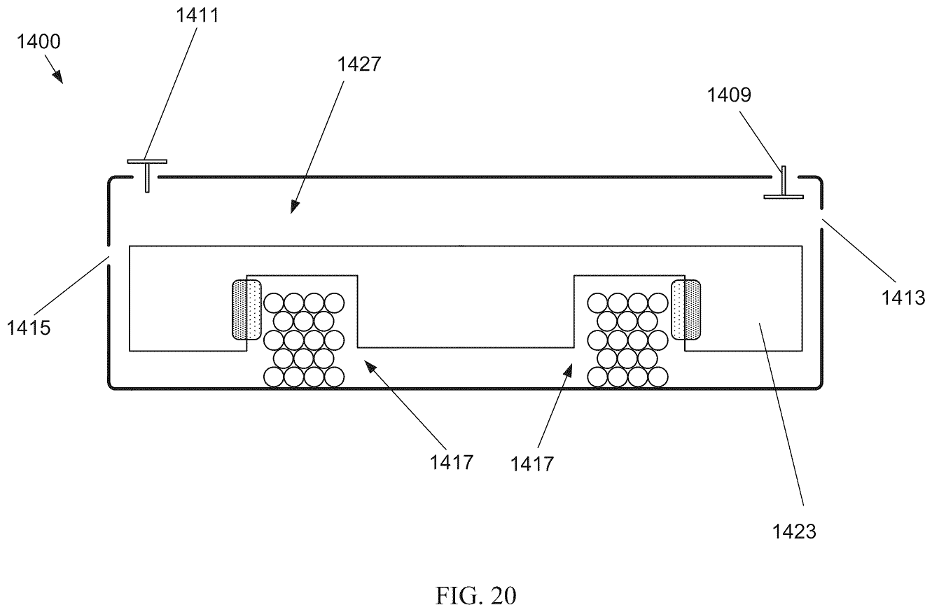

FIG. 20 is a schematic view of an alternate configuration of the breast pump device 1400, shown in the fourth position. In the example of FIG. 20, the device 1400 includes a coil 1417, a piston 1423 a second chamber 1427, a first valve 1409, a second valve 1411, a first port 1413, and a second port 1415. The O-rings on the piston 1423v are not drawn to simplify the schematic view. The example of FIG. 20 functions equivalently to the example breast pump device 1300 described with reference to FIGS. 15-19, above.

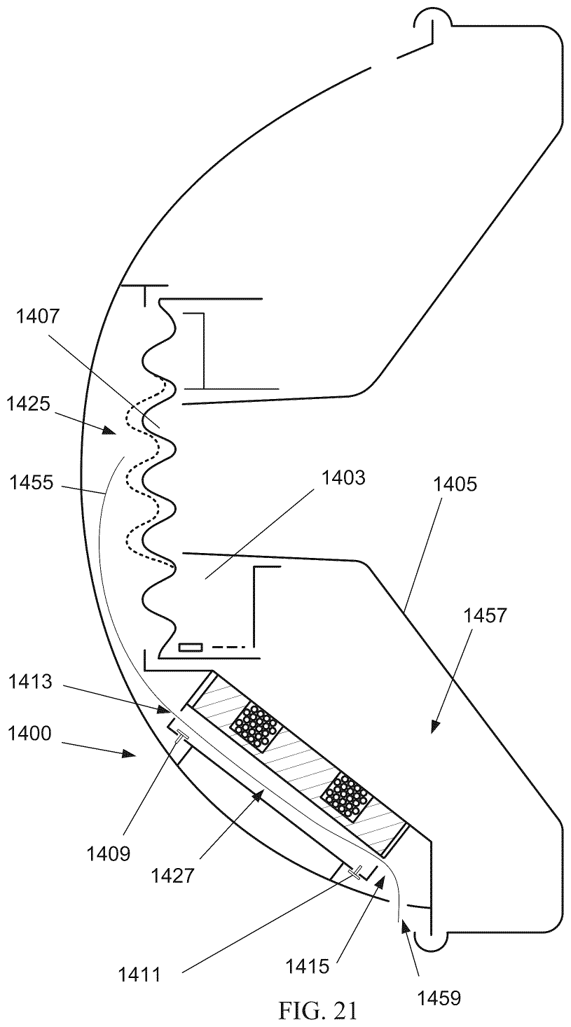

FIG. 21 is a schematic view of an example implementation of the breast pump device 1400 of FIG. 20 with a diaphragm 1407 and funnel 1405. In the example of FIG. 21, the funnel 1405 includes an interior cavity 1457 in which the breast pump device 1400 of FIG. 20 is placed. The funnel 1405 also includes a collection receptacle 1403 and a diaphragm 1407, separating the interior cavity 1457 of the funnel 1405 from the exterior of the funnel 1405. The breast pump device 1400 is positioned within the interior cavity 1457 of the funnel 1405 so that the side of the breast pump device 1400 with the second port 1415 and second valve 1411 are sealed off from the rest of the interior cavity 1457 but are able to communicate with the exterior space through an opening 1459 in the funnel 1405.

The other side of the breast pump device 1400 includes the first port 1413 and the first valve 1409, each of which are sealed off from the rest of the interior cavity 1457 of the funnel 1405, but are able to communicate with a portion of the interior cavity 1457 of the funnel 1405 that functions as the first chamber 1425. The first chamber 1425 is adjacent to the diaphragm 1407 so that pressure changes in the first chamber 1425 effect deviations in the diaphragm 1407. As the breast pump device 1300 creates pressure differentials, causes deviations in the diaphragm 1407, and generates suction by the funnel 1405, a woman's breast, sealed within an opening of the funnel 1405, can express breastmilk, which flows into the collection receptacle 1403.

After collecting the breastmilk, the breast pump device 14000 can release the pressure by moving back to position three (depicted in FIG. 21). When in position three, the breast pump device 1400 allows air to flow through from the exterior space, through the second port 1415, into the second chamber 1427, through the first port 1413, and into the first chamber 1425. The opening of the ports 1413, 1415 allows the pressure within the first and second chambers 1425, 1427 to neutralize, which results in the diaphragm 1407 relaxing back to its neutral position. The airflow path from the exterior space into the first chamber 1425 is illustrated by line 1455.

In some embodiments, the vibration produced by the breast pump device 1400 can be tuned by a user to accomplish a desired intensity. In some examples, the vibration functionality can be turned on or off by the user through the use of a control mechanism, such that the timing and frequency of the vibrations could create different amplitudes and/or other operating parameters of the vibration while also giving the user ability to turn the vibration function off completely. The control mechanism could comprise a complex of a PCBA—with or without: power, voltage, current and/or pressure sensors or other sensors, and/or power circuits with AC or DC power including wall connected power and/or battery power.

This tunability could also be adjusted by the firmware of the system such that it compensates for differences in the vacuum release speed at different vacuum levels, to maintain a constant vibration amplitude of the vibration regardless of the vacuum level. For example, at low vacuum, it might require 0.1 seconds to release 10 mmHg, while at high vacuum, it might only require 0.05 second to release 10 mmHg. In other embodiments, it may be desired to anti-compensate or not-compensate for this different vacuum release speed or rush in air speed during the vibrations being created such that vibration amplitudes could differ along the suction curve of decreasing vacuum.

The vacuum drive system could also be tuned such that it could operate more strongly or less strongly to control the vibration rate and provide additional control over the desired performance characteristics of the system. Using a combination of operating parameters associated with the drive system, the user (and possible the drive system itself) could manipulate the drive system to provide for a wide variety of unique waveforms of vacuum phases and vibrations, including turning off the vibrations completely.

In some examples, the breast pump device 1400 could store certain operating parameters locally or on a profile remotely (e.g., in the cloud) relating to the user's preferences, such as the vibration frequency, amplitude of the vibration, if vibration is present or should not be present during stimulation and/or expression modes or none of the modes, random vibration patterns or consistent patterns, and any other variations in combination thereof. These user preferences could be displayed on a mobile device in an application or system if linked via a connection, Bluetooth, radio frequency, Wi-Fi, or other data transmission system that would provide input and data from one device to another.

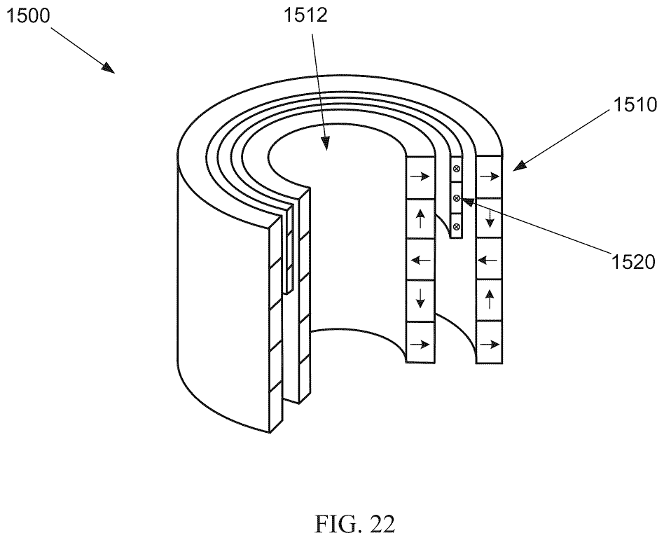

The breast pump device 1400 can use a positional sensor, such as a hall effect sensor, to determine the position of the piston in order to achieve the four positions. Another embodiment of the breast pump device 1400 shown in FIG. 22 can use a voice coil 1500 with a Halbach array 1510, 1512 to create different magnetic zones to eliminate the need for a positional sensor. In conjunction with the Halbach array, a bobbin can have one or more coils 1520, where each coil can have windings in the clockwise direction (CW), counterclockwise direction (CCW) or in both directions.

A microcontroller can control the direction of the current within each coil programmatically to lock-in to the boundary of the magnetic field generated by the Halbach array, thereby removing the need for a positional sensor. In FIG. 22, the three coils 1520 will have current flowing in the CCW, CCW, CW for position 1, CCW, CW, CW for position 2, CW, CW, CCW for position 3, and CW, CCW, CCW for position 4.

The voice coil can be a moving magnet design, as depicted in FIGS. 15-19, or a moving coil design. The benefit of a moving magnet can be that the heat generated by the coil can be dissipated by the stationary bobbin. Also, the moving mass can act as a source of mechanical vibration, thus the number of required positions can be reduced. However, the force required to move the mass can be significantly larger than that of a moving coil.

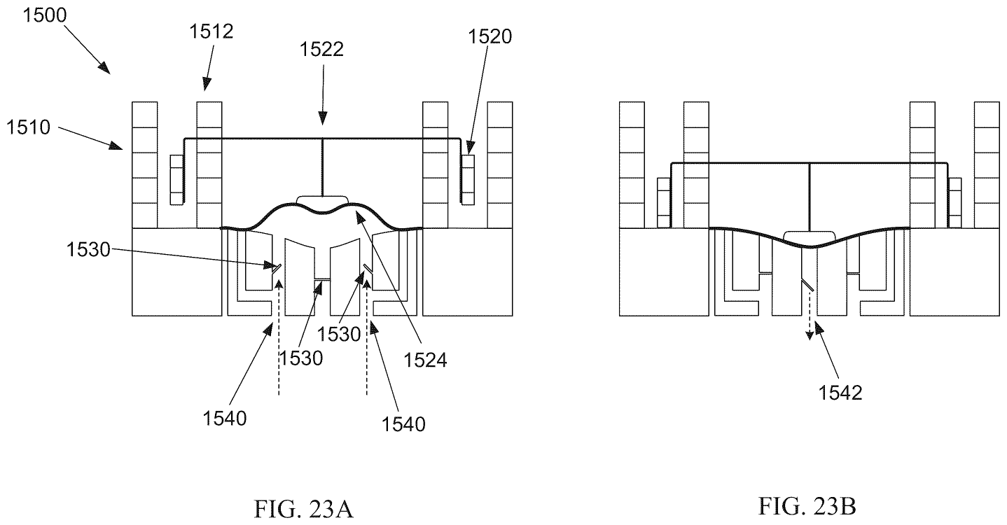

Now referring to FIGS. 23A-25B, the breast pump device can be designed to not use a piston. A diaphragm can replace the piston to increase reliability and decrease the probability of a leak. The diaphragm can be designed such that it also acts as a controllable valve. The diaphragm can be designed to enable vacuum, vibration and vacuum release (venting).

In one possible embodiment, the diaphragm can be connected physically to a bobbin which contains an electromagnetic coil wrapping that can be actuated through the action of a second magnetic force generating element such as a magnet or a second electromagnetic coil. The bobbin may be attached through a physical connector or friction reducing element such as a bearing which may also serve to limit the degrees of movement of the bobbin to the desired alignment.

The diaphragm may be positionally actuated with the magnetic force being applied to the system which actuates the diaphragm through physical force or which could actuate the diaphragm through electromagnetic force in alternative embodiments where the diaphragm contains a coil or a magnetic internal to at least a portion of the diaphragm. The position of the diaphragm could be actuated such that the diaphragm could close an open port or open a port as desired to create one or more actions of the pump system including but not limited to vibration through the action of backpressure, vibration through the action of partial vacuum release at specific times, vacuum accumulation through the expelling of air, vacuum release through the accepting of air into the system from an alternative area such as but not limited to ambient environment.

FIGS. 23A-23B show one example vacuum operation. A bobbin 1522 is connected to three coils 1520 and also to a diaphragm 1524. The bobbin 1522 is moved to position 2 (FIG. 23B) from position 1 (FIG. 23A) by controlling the current in the three coils 1520 such that the bobbin 1522 is self-positioned with the Halbach arrays 1510 and 1512. As the bobbin 1522 moves from position 1 to position 2, air is pulled in from the inlet ports 1540 through the one-way valves 1530, thus creating a vacuum on the breast (not shown). The next phase is to expel the air back to the outside. The bobbin 1522 is moved from position 2 to position 1 by adjusting the current in the three coils 1520. The air is then displaced through the one-way valves 1530 and out to atmosphere through the outlet port 1542.

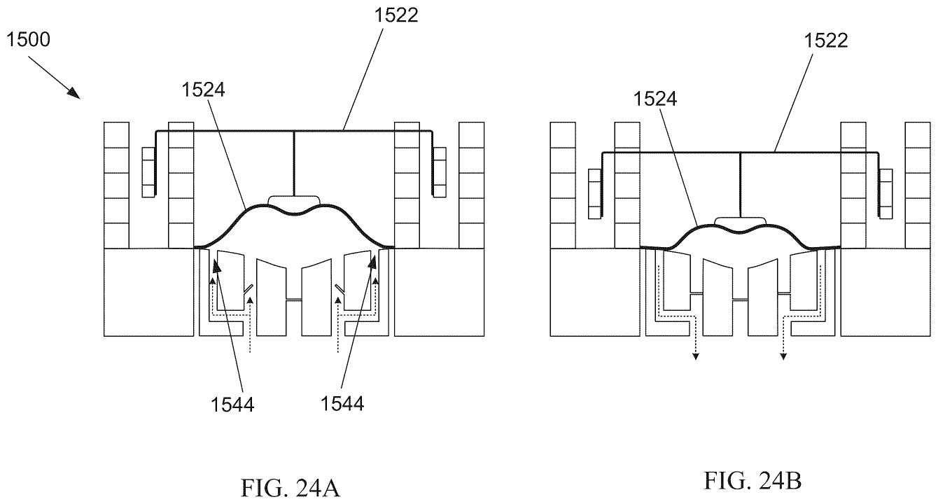

FIGS. 24A-24B illustrate the vibration mode. As the bobbin 1522 is moved from position 2 (FIG. 24A) to position 3 (FIG. 24B), the diaphragm 1524 is pulled up to open bypass port 1544. The bypass port 1544 allows the air to be pulled in from the breast but also expelled back to the breast, creating the vibration sensation on the breast.

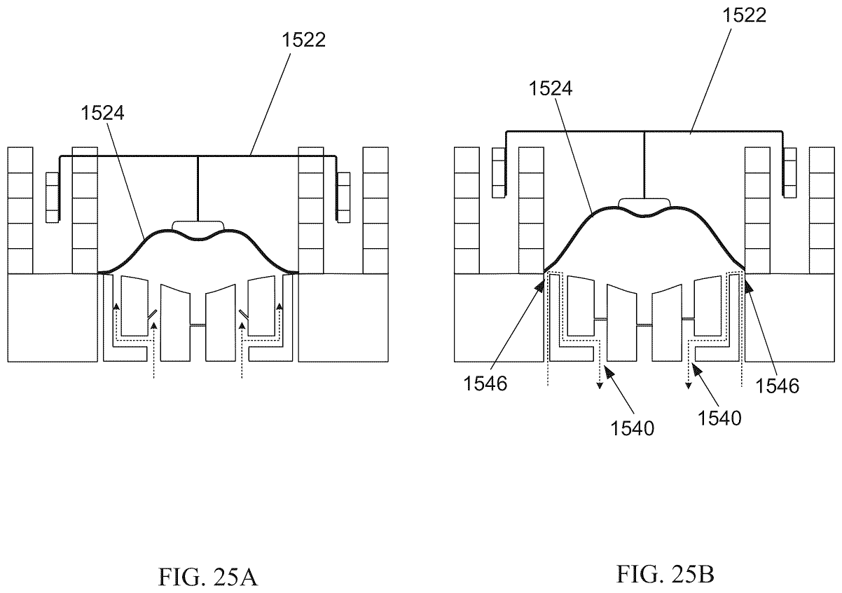

FIGS. 25A-25B illustrate the release mode. As the bobbin 1522 is moved further up from position 3 (FIG. 25A) to position 4 (FIG. 25B), the diaphragm 1524 exposes the internal chamber to the release port 1546 which is connected to the outside. This allows the vacuum present in the inlet port 1540 to equalize to atmosphere pressure via the release port 1546, thus releasing the vacuum present on the breast.

The release mode can also be used to generate vibration, by controlling the amount of vacuum released through the release port 1546. For example, the bobbin 1522 can alternate between position 1 and 2 for 10 cycles followed by 1 and 4 for one cycle, and repeat until the target vacuum is reached. Then the bobbin 1522 is moved to position 4 until the vacuum has been fully released.

As previously noted, the voice coil actuator can provide one or more functions, including but not limited to vacuum generation, vibration, and release of vacuum. In some examples provided herein, the voice coil actuator provides all three functions. In other examples, the voice coil actuator provides one or two functions and another motor (e.g., a solenoid) provides the other function(s).

For example, referring now to FIG. 26, in an alternative embodiment, a breast pump system 1600 may include a vacuum motor 1601, a solenoid 1603, and a flange assembly 1604, all connected by a tube 1606 or other suitable connector. The vacuum motor 1601, which is a voice coil actuator in this example, provides the main source of vacuum for driving the breast pump system 1600 and providing suction to the flange assembly 1604. The solenoid 1603 generates the vibrations for the vibratory waveform. Many other configurations are possible.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

What is claimed is:

1. A vacuum motor device for facilitating milk extraction from a breast of a user, the vacuum motor device comprising:
a first chamber;
a second chamber coupled to the first chamber; and
a voice coil motor configured to cause air to flow into the second chamber and out of the first chamber during a breast pumping cycle to create suction for extracting the milk.

2. The vacuum motor device of claim 1, wherein the voice coil motor includes a coil, a magnet, and a diaphragm.

3. The vacuum motor device of claim 1, wherein the voice coil motor includes a coil, a magnet, and a piston.

4. The vacuum motor device of claim 3, wherein a current flowing through the coil in a first direction generates a magnetic field, and wherein the magnetic field pushes the magnet and the piston into the coil.

5. The vacuum motor device of claim 4, wherein the current flowing through the coil in a second direction changes the magnetic field to push the magnet and the piston out of the coil.

6. The vacuum motor device of claim 1, further comprising a first directional valve positioned to allow the air to flow between the first chamber and the second chamber, and a second directional valve to allow the air to flow between the second and an ambient environment.

7. The vacuum motor device of claim 1, further comprising a first port configured to allow the air to flow between the first chamber and the second chamber when a piston or bobbin is in a first position.

8. The vacuum motor device of claim 7, wherein the air flowing through the first port creates vibratory waveform to the breast pumping cycle.

9. The vacuum motor device of claim 8, wherein the vibratory waveform is controllable by the user.

10. The vacuum motor device of claim 9, further comprising a feedback control mechanism configured to tune the vibratory waveform.

11. The vacuum motor device of claim 7, further comprising a second port configured to allow the air to flow between the first chamber, the second chamber, and an ambient environment when the piston or bobbin is in a second position.

12. A method for facilitating milk extraction from a breast of a user, the method comprising:
providing a first chamber;
providing a second chamber coupled to the first chamber; and
providing a voice coil motor configured to cause air to flow into the second chamber and out of the first chamber during a breast pumping cycle to create suction for extracting the milk.

13. The method of claim 12, wherein the voice coil motor includes a coil, a magnet, and a piston or diaphragm.

14. The method of claim 13, wherein a current flowing through the coil in a first direction generates a magnetic field, and wherein the magnetic field pushes the magnet and the piston or the diaphragm into the coil.

15. The method of claim 14, wherein the current flowing through the coil in a second direction changes the magnetic field to push the magnet and the piston or the diaphragm out of the coil.

16. The method of claim 12, further comprising providing a first directional valve positioned to allow the air to flow between the first chamber and the second chamber, and a second directional valve to allow the air to flow between the second and an ambient environment.

17. The method of claim 12, further comprising providing a first port configured to allow the air to flow between the first chamber and the second chamber when a piston or diaphragm is in a first position.

18. The method of claim 12, wherein the voice coil motor includes a coil, a magnet, and a piston or diaphragm.

19. The method of claim 18, wherein a current flowing through the coil in a first direction generates a magnetic field, and wherein the magnetic field pulls the coil and the piston or the diaphragm into the magnet.

20. The method of claim 19, wherein the current flowing through the coil in a second direction changes the magnetic field to push the coil and the piston or the diaphragm out of the magnet.
